(19) United States
(12) Patent Application Publication
(10) Pub. No.: US 2021/0402160 A1
(43) Pub. Date: Dec. 30, 2021

(54) BALLOON CATHETER

(71) Applicant: TriReme Medical, LLC, Pleasanton, CA (US)

(72) Inventors: Eitan Konstantino, Orinda, CA (US); Tanhum Feld, Moshav Merhavya (IL); Gary Binyamin, Berkeley, CA (US); Guillermo Piva, San Ramon, CA (US)

(21) Appl. No.: 17/445,696

(22) Filed: Aug. 23, 2021

Related U.S. Application Data
(63) Continuation-in-part of application No. 17/049,827, filed on Oct. 22, 2020, now abandoned.
(60) Provisional application No. 62/662,160, filed on Apr. 24, 2018.

(51) Int. Cl. A61M 25/10

(52) U.S. Cl. CPC A61M 25/1029; A61M 25/1002; A61M 2025/1004; A61M 2025/105; A61M 2025/1031

(57) ABSTRACT

A balloon catheter and methods of using same are provided. The balloon catheter can include an expandable structure mounted over a balloon coated with a composition. The expandable structure includes a plurality of axial struts crossing a plurality of radially-expandable rings for constraining the balloon such that isolated balloon regions can protrude through openings in the expandable structure when the balloon is inflated. The balloon catheter can be configured to maximize scraping of the composition from the surface of the balloon by the struts of the expandable structure during balloon inflation.

### INCORPORATION BY REFERENCE TO ANY PRIORITY APPLICATIONS

This application is a continuation-in-part of U.S. application Ser. No. 17/049,827, filed Oct. 22, 2020, which is a national phase application of PCT/US2019/028481, filed Apr. 22, 2019, which claims the priority benefit of U.S. Application No. 62/662,160, filed Apr. 24, 2018, all of which are hereby incorporated by reference in its entirety herein.

Any and all applications for which a foreign or domestic priority claim is identified in the Application Data Sheet as filed with the present application are hereby incorporated by reference under 37 C.F.R. § 1.57.

### BACKGROUND

### Field

The present application relates to a drug coated balloon and methods of using and manufacturing the same.

### Description of the Related Art

Vascular stenosis is a common disease with variable morbidity affecting mostly men and women older than 50 years. Vascular stenosis is characterized by narrowing of a blood vessel lumen (typically an artery) due to intraluminal deposits of plaque material (typically fat and calcium).

Percutaneous transluminal angioplasty (PTA) is a procedure in which a thin, flexible tube called a catheter is inserted through an artery and guided to the place where the blood vessel is narrowed. When the tube reaches the narrowed artery, a small balloon at the end of the tube is inflated such that the pressure from the inflated balloon forces the plaque material against the wall of the artery to open the vessel and improve blood flow.

Damage to the vessel wall resulting from balloon inflation can lead to re-narrowing of the blood vessel in a process termed restenosis.

Drug-Coated Balloon (DCB) PTA is similar to plain balloon angioplasty procedurally with the addition of an anti-proliferative medication delivered from the balloon to help prevent restenosis.

### SUMMARY

The drug (e.g., Paclitaxel and Sirolimus) in DCBs may be applied along with a carrier or matrix to the balloon external surface before the balloon is folded or following folding using techniques such as dipping or deposition. In order to provide predictable dosing to the treated area, care should be taken that the drug is evenly distributed over the balloon surface contacting the lesion.

In order to maximize drug delivery to the treated site independent of the anatomy, a DCB should exhibit minimal drug loss during transit and maximal release of the drug at the treated site.

Conventional DCBs are susceptible to a significant amount of drug coating loss during guiding to the target site (transit) and typically inflate unevenly while causing trauma and dissections to the vessel wall, resulting in delivery of only a portion of the drug in a non-uniform manner. The amount of drug loss during transit can range from 20% to 85% of the total dose coated on the balloon and actual drug delivery to the vessel wall is on the order of 2% to 40% of the total dose. In addition, drug distribution at the target site is typically not uniform due to drug losses caused by transit and balloon inflation. Furthermore, since drug delivery is passive, it is in direct relationship to the time required to maintain an inflated balloon at the treatment site (residence) as well as the size of the balloon and forces applied thereby to the vessel wall. As such, DCBs oftentimes require prolonged residence times of up to 2 minutes.

There is thus a need for, and it would be highly advantageous to have, a drug coated balloon configured for minimizing drug loss during transit and maximizing drug delivery at the treatment site.

Embodiments of the present application relate to a balloon catheter having an expandable structure mounted over the balloon and being configured for constraining balloon inflation and facilitating release of a drug coating thereof.

Some aspects of the disclosure are directed to a balloon catheter comprising an expandable structure mounted over a balloon, the expandable structure including a plurality of axial struts crossing a plurality of radially-expandable rings for constraining the balloon such that isolated balloon regions protrude through openings in the expandable structure when the balloon is inflated. Each of the axial struts has a multi-sided, e.g., four-sided, cross section and/or rounded corners. The radius of curvature of the rounded corners may be selected from a range of 0.01 mm to 0.05 mm.

Some aspects of the disclosure are directed to a balloon catheter comprising an expandable structure mounted over a balloon, the expandable structure including a plurality of axial struts crossing a plurality of radially-expandable rings for constraining the balloon such that isolated balloon regions protrude through openings in the structure when the balloon is inflated. The balloon may include a plurality of pleated folds having a fold overlap that is 50% to 80% of a distance between adjacent struts.

Some aspects of the disclosure are directed to a balloon coated with a composition and an expandable structure mounted over the balloon. The expandable structure may include a plurality of axial struts crossing a plurality of radially-expandable rings to form a plurality of openings. The balloon catheter is configured to transition between a collapsed configuration and an expanded configuration. In the collapsed configuration, the balloon includes a plurality of pleated folds beneath the expandable structure. In the expanded configuration, isolated balloon regions protrude through the openings in the expandable structure. The expandable structure is configured to scrape the composition from the balloon as the balloon catheter transitions from the collapsed configuration to the expanded configuration.

In any of the above mentioned balloon catheters, a length of overlap of each of the plurality of pleated folds may be less than a distance between adjacent axial struts of the plurality of struts.

In any of the above mentioned balloon catheters, the balloon may be coated with a composition, such as an anti-proliferative drug.

In any of the above mentioned balloon catheters, the balloon may include at least two and/or less than or equal to six pleated folds in an uninflated state. The pleated folds may unfold during inflation of said balloon to scrape composition against each struts.

In any of the above mentioned balloon catheters, a distance between adjacent struts of may be selected from a range of 0.4 mm to 1.1 mm when said expandable structure is in a non-expanded state.

In any of the above mentioned balloon catheters, each strut may have a width selected from a range of 70 to 90 microns and/or a height selected from a range of 80 to 120 microns.

Some aspects of the disclosure are directed to a method of treating a stenosed vessel comprising delivering the balloon catheter described herein to a region of stenosis in the vessel, inflating a balloon of the balloon catheter to thereby form isolated balloon regions protruding through openings in the expandable structure and scrape off the composition to thereby treat the stenosed vessel.

Typical angioplasty balloons are cylindrical in shape when inflated and comprised of a single material. These symmetric, single material structures are conducive to coating. However, specialty balloons, such as those described herein, may be non-cylindrical and have a relatively complex geometry and/or comprise multiple materials. The processes described herein provide a coating on these specialty balloons. The coating may include one or more therapeutic layers that are intended to be retained during delivery to stenotic vasculature and transferred to the vessel wall during inflation. Although certain balloon designs are described herein, the method may also be applied to other non-cylindrical and/or multi-material balloons, including but not limited to, cutting balloons, woven balloons, balloon-in-balloon, scoring balloons, tapered balloons, ostial balloon, or low-trauma balloons.

Certain aspects of the disclosure are directed toward methods of manufacturing any of the drug-coated balloon catheters described herein. The method may include fixedly mounting an expandable structure, such as a nitinol expandable structure, over a balloon. The method may include surface treating the balloon using one or more processes. For example, the balloon may be surface treated by spraying carbon dioxide on a surface of the balloon and/or applying plasma to the surface of the balloon. Surface treating may take place prior to or following the step of mounting the expandable structure over the balloon. The method may also include coating the balloon and/or expandable structure with one or more layers of a therapeutic composition. The composition may include at least one active pharmaceutical ingredient and at least one excipient. Prior to coating the balloon, the balloon may be inflated with the expandable structure mounted over the balloon. The balloon may be fully inflated to its indicated pressure. The inflated balloon may form isolated balloon regions protruding through openings in the expandable structure such that the coating may be applied to the isolated balloon regions. After coating, the balloon may be deflated to form a plurality of pleated folds beneath the expandable structure.

Unless otherwise defined, all technical and scientific terms used herein have the same meaning as commonly understood by one of ordinary skill in the art. Although methods and materials similar or equivalent to those described herein can be used in practice or testing, suitable methods and materials are described below. In addition, the materials, methods, and examples are illustrative only and not intended to be limiting.

### BRIEF DESCRIPTION OF THE DRAWINGS

The balloon catheters are herein described, by way of example only, with reference to the accompanying drawings. With specific reference now to the drawings in detail, it is stressed that the particulars shown are by way of example and for purposes of illustrative discussion only, and are presented in the cause of providing what is believed to be the most useful and readily understood description of the principles and conceptual aspects of the present disclosure. In this regard, no attempt is made to show structural details of the embodiments in more detail than is necessary for a fundamental understanding of the embodiments, the description taken with the drawings making apparent to those skilled in the art how the several forms of the embodiments may be embodied in practice.

FIGS. 1A-1D illustrate a balloon catheter in various states of inflation.

FIGS. 2A-2B illustrate several strut profiles suitable for use in the expandable structure of the balloon catheter.

FIGS. 3A-3E illustrate balloon unfolding during inflation.

FIGS. 4A-4D illustrate strut distance to fold overlap in a 3 pleat balloon.

FIGS. 5A-5B illustrate strut distance to fold overlap in a 6 pleat balloon.

FIG. 6 illustrates a flow chart of a coating process.

### DETAILED DESCRIPTION

The present disclosure relates to a drug coated balloon which can be used to effectively treat vascular stenosis. Specifically, the drug coated balloon can be used to open blocked vessels and deliver an anti-proliferative drug to a site of treatment in an efficient and effective manner.

The principles and operation of the present disclosure may be better understood with reference to the drawings and accompanying descriptions.

It should be understood that the present disclosure is not limited in its application to the details of construction and the arrangement of the components set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting.

Drug coated balloons (DCBs) were developed in order to treat restenosis following angioplasty. Although such balloons are efficacious in reducing the incidence and severity of restenosis, present designs still suffer from several limitations including loss of drug during transit and incomplete drug transfer to the artery wall. The balloon catheters described herein minimize the aforementioned limitations.

The balloon catheter includes a balloon having an expandable structure [also referred to herein as “an expandable constraining structure (CS)” ] mounted there around and fixedly attached to one or both ends to the catheter (see, for example, U.S. Publication No. 20140066960 which is fully incorporated by reference herein).

In the non-expanded state, the balloon is folded (e.g., two to six folded pleats) with the expandable structure collapsed over the folded balloon.

In the deployed (expanded) state, the expandable structure of the present balloon catheter has a final diameter that is smaller than that of the fully inflated balloon. While the struts and rings of the expandable structure limit balloon diameter at points of contact (creating depressions in the balloon surface), the openings between the struts and rings do not, and as such, isolated balloon regions protrude from these openings in the expandable structure when the balloon is fully inflated. Such a unique configuration protects the vessel wall from the effects of balloon unfolding and uneven inflation, while also enabling application of localized forces to a discrete plaque region.

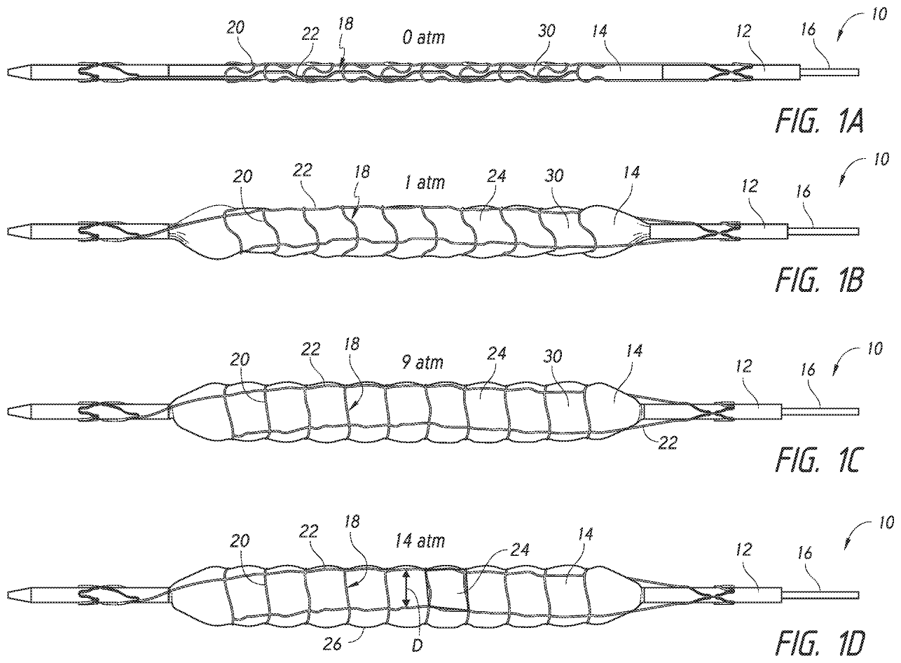

As shown in FIGS. 1A-1D, there is provided a balloon catheter having an expandable structure mounted over the balloon. The balloon catheter can be configured for use in any biological vessel where release of a composition for treatment or diagnostics is desired (e.g., urinary vessels, ducts, GI tract etc.). One specific use for the present balloon catheter is in an angioplasty procedure (e.g., coronary, peripheral, neurological, etc.) on a human subject.

The balloon is coated with one or more layers of a composition that can include, for example, a suitable solvent or mixture of solvents, a carrier (e.g., binder), an excipient and one or more active pharmaceutical ingredients having anti-inflammatory, cytostatic, cytotoxic, antiproliferative, anti-microtubule, anti-angiogenic, anti-restenotic (anti-restenosis), fungicide, antineoplastic, antimigrative, athrombogenic and/or antithrombogenic activity. The active ingredient can be in the form of particles (e.g., nanoparticles) or provided in free form in the coating.

The solvents used are typically volatile or semi-volatile, allowing for distribution over the expandable surface of the catheter assembly. Solvent combinations are intended to facilitate deposition, both spatially over the surface and in the correct form for passive uptake during inflation. Alternatively, a solvent system can be applied containing the drug in order to distribute spatially and a second solvent system applied to achieve the correct form. An example of solvents used includes mixtures of acetone, tetrahydrofuran, mono-alcohols (e.g., methanol, ethanol, isopropanol), and water. Examples of active pharmaceutical ingredients include one or more of the following: taxanes (e.g., paclitaxel, docetaxel, protaxel), mTor inhibitors (e.g., sirolimus, everolimus, zotarolimus, biolimus), cilostazol, and statins. Final concentrations of the active pharmaceutical ingredient is between 0.5 μg/mm² to 25 μg/mm², and for example between 1-10 μg/mm².

Excipient examples that may be included are urea, shellac, citrate ester, polysorbate/sorbitol, propyl gallate, nordihydroguaiaretic acid, resveratrol, and butylated hydroxy toluene. The loading of the transport enhancer is between 3-100% of the weight of the drug. Polymers can act as carriers (e.g., binders), which can have hydrophilic, hydrophobic, or amphiphilic characteristics. These can be durable or biodegradable molecules. Some carriers include poly(ethylene glycol), poly(vinyl alcohol), hydroethyl cellulose, methyl cellulose, dextran, and poly(vinyl pyrrolidone).

A specific example of coating is a solvent mixture of acetone, ethanol, and water containing paclitaxel and propyl gallate at a ratio of 2:1 by weight. A specific volume of the solution is applied to the expandable portion of the balloon catheter to achieve a paclitaxel dose density of 3 μg/mm². The coating is formed upon drying of the solvents.

The expandable structure includes a plurality of rings crossing a plurality of struts to form a cage like structure trapping the balloon. Both rings and struts can be expanded to a final diameter and length (respectively) by including linearizable regions such as zigzag or s-wave regions within the rings/struts. The expandable structure can be fixedly attached to the catheter shaft at one end only with the other end being mounted over the shaft and slidable thereagainst. Such a configuration enables the expandable structure to shorten during inflation to accommodate for radial expansion. In other configurations, the expandable structure can be fixedly attached to the catheter shaft on opposing sides of the balloon.

The profile of the struts (and optionally rings) is specifically configured in order to facilitate drug scraping/wiping from the surface of the balloon when the balloon inflates and unfolds. Scraping/wiping can release the drug from the surface of the balloon or it can redistribute (concentrate) the drug along regions on the surface of the balloon.

As a pleated balloon unfolds, the pleats shorten and the balloon surface moves circumferentially (in a balloon folded using the concentric technique). Since the present balloon catheter includes struts and rings mounted over the balloon and in contact therewith, the balloon surface moves against the struts (the inner surface and edge of the strut) as the balloon inflates and unfolds.

Thus, any coating on the balloon surface is effectively scraped (wiped) by the struts (and optionally by the rings) as the balloon inflates and unfolds.

Thus, the present balloon catheter is advantageous in that the expandable structure protects the balloon coating from loss during transit and acts as a scrape to facilitate release of the drug coating at the site of treatment.

Two opposing needs were considered when designing the profile of the struts of the present balloon catheter. Scraping can be enhanced by a strut profile that displays a sharp edge to the moving balloon surface. Such an edge profile can effectively lift and separate the coating from the balloon surface. However, a sharp edge can also damage the balloon surface and lead to balloon rupture. In order to maximize both scraping and protect the balloon from rupture during unfolding, the strut profile may include four sides (e.g., square, rectangular, trapezoid) with rounded edges having a radius of curvature of 10 to 40 microns. The struts can have a width selected from a range of 70 to 90 microns and a height selected from a range of 80 to 120 microns and can be electropolished.

Such dimensions and profile ensure that the struts provide the necessary stability to the expandable structure (to constrain the balloon at high pressures), prevent balloon rupture during inflation while effectively scraping the balloon surface to present most, if not all, of the coating for transfer during inflation. Since the pillows formed following inflation concentrate a radial outward force applied by the balloon on the vessel wall, the drug distributed over the balloon surface following scraping is delivered through such direct contact.

As is mentioned hereinabove, present DCBs are limited by drug loss during transit. Although a coating that more strongly adheres to the balloon surface can be used to minimize such loss, strongly-bound coatings require longer balloon residence times to effectively release the required dose at the site of treatment.

Since the present balloon catheter employs a scraping mechanism such a tradeoff between drug binding and drug release is not a limitation thereof.

As such, the present balloon catheter can include coatings that are strongly bound to the balloon surface to further minimize drug loss during transit.

Such coatings can include binding agents such as hydrophilic, hydrophobic, or amphiphilic polymers. These can be durable or biodegradable molecules.

Binders can be mixed within the layer containing the active pharmaceutical ingredient or they can be used as a base layer, a cover layer or more than one layer.

Prior to inflation, the balloon is folded underneath the expandable structure. Drug coating is disposed on the external surface of the balloon (and sometimes at least partially over the structure) along at least a portion of its working length, e.g., the surface in between the balloon tapers. Balloon tapers may or may not have drug coating.

A standard balloon catheter typically travels 1.0 m to 1.5 m through the vascular during delivery, from the access site to the treatment site. The balloon may be folded to a smaller diameter in order to allow delivery thru tight vascular anatomy. For example balloons with nominal inflated diameter of 2 mm to 6 mm will have a folded diameter of 0.7 mm to 1.5 mm. However, despite folding, a significant part of the outer surface of the balloon and drug coating is exposed to the blood and vessel wall during delivery. Contact and friction between the balloon external surface and the vessel wall are especially significant when going through tortuous anatomy that forces the balloon against the vasculature. Delivery of a folded balloon, without a constraining structure, over a bend or a curved segment will open up the folds of the balloon since the folds are not protected and the part of the balloon closer to the inner radius of the bend covers a shorter distance than the part of the balloon closer to the outside radius of the bend. Those elements lead to significant exposure and drug loss during delivery. Loss of drug prior to inflation within the lesion results in reduced or unpredictable therapeutic coverage that should have been delivered at the occlusion site on one hand, and un-desired systemic drug and particulates release to the patient body that could have arbitrary or harmful impact, such as occlusion of small arteries and toxicity.

Since the present balloon catheter includes an expandable structure disposed around the balloon, the coating is protected during delivery thus minimizing loss to the dose available prior to deployment at the target site. In addition, the expandable structure compresses the balloon and prevents unfolding thereof when going through a vessel.

During delivery, the balloon is deflated and folded and the expandable structure covers approximately 10% to 50% of the exposed surface of the balloon. When the device is inflated to nominal pressure, e.g., between 8 ATM to 10 ATM, the space between longitudinal adjacent struts increases such that the expandable structure covers approximately 5% to 20% of the working length surface thereby allowing the distributed drug released by scraping of the struts to contact the vessel wall and diffuse thereinto.

The distance between two adjacent struts of a nominally inflated balloon divided by the distance between two adjacent struts of a folded balloon, ranges from 1.7 to 5.5 for balloons with nominal diameters of 2.0 mm to 4.0 mm using four longitudinal struts and 2.4 to 5.5 for balloon of 4.5 mm to 7 mm with six longitudinal struts.

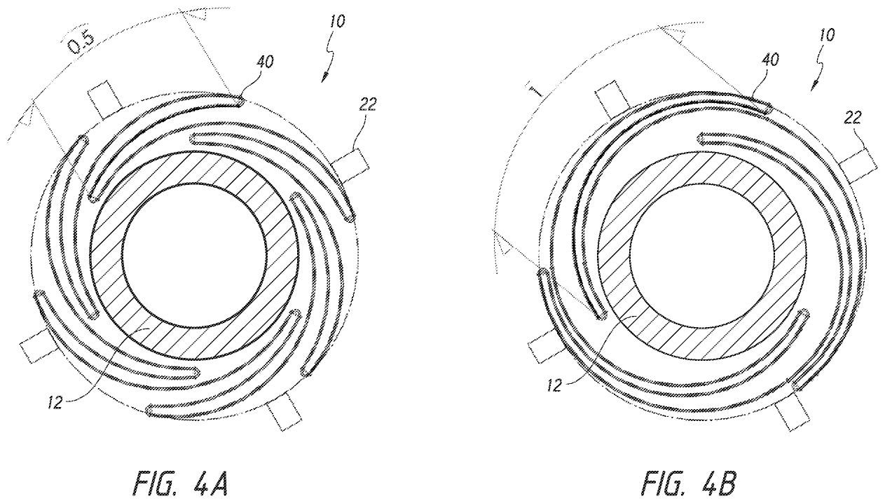

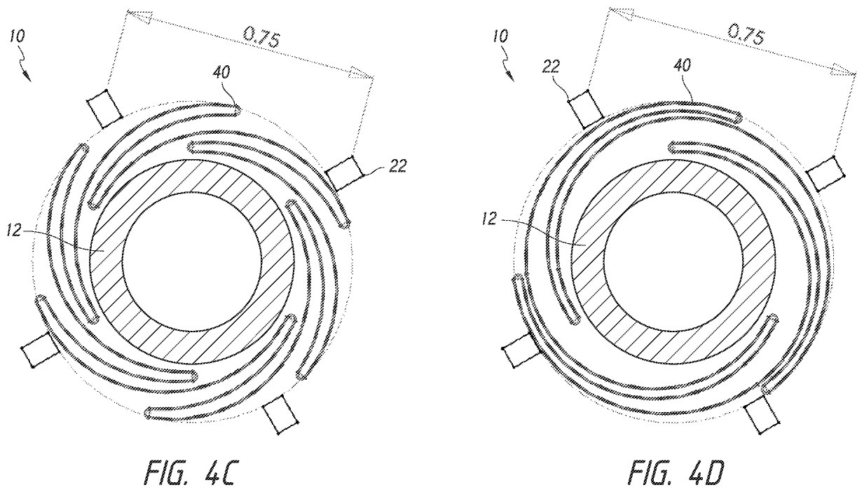

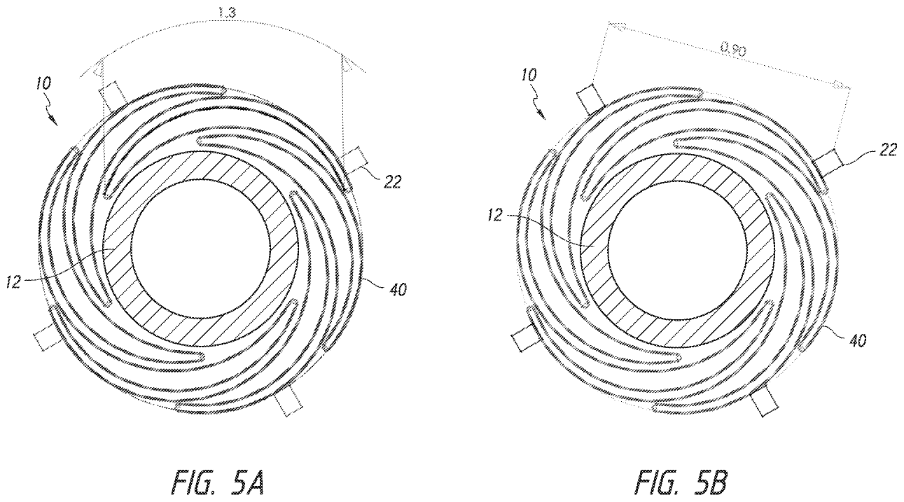

Drug scraping and release can be optimized by selecting the distance between adjacent struts and/or the ratio between fold size (length of overlap of fold over balloon surface) (see, e.g., FIGS. 4A, 4B, and 5A) and distance between adjacent struts (see, e.g., FIGS. 4C, 4D, and 5B). The fold size may be 50% to 80% of a distance between adjacent struts. The ratio between fold size and between adjacent struts can be between 1:0 and 1:1.5 or between 1:0.75 and 1:1.5.

If the distance between adjacent struts is larger than the fold overlap, scraping may be less effective scraping along the struts. A small number of pleats for a given diameter will result in longer pleats and therefore more rotation when the balloon unwraps. It is therefore advantageous to have a low number of pleats in order to enhance scraping. On the other hand, a small number of pleats may apply high torsional forces on the expandable structure and cause it to break so the optimal number has to be considered taking into consideration the distance between adjacent struts as it compared to the pleat length. The number of pleats may be greater than or equal to two and/or less than or equal to six.

For balloons with diameters ranging from 2 mm to 4 mm (inflated) the distance between two adjacent struts can be selected from a range of about 0.4 mm to 0.8 mm and the length of the overlap of the pleats can be about 0.2 mm to 0.8 mm if six pleats are used and about 0.4 mm to 1.6 mm if three pleats are used. Such a configuration can enhance scraping against the struts (and rings).

In some configurations, the length of the fold overlap may be greater than the distance between adjacent struts. For example, a balloon with a diameter of 3 mm and 3 pleats, the ratio between fold overlap and the distance between adjacent struts can be about 1:0.75.

For balloons with diameters ranging from 4.5 mm to 7 mm the distance between two adjacent struts can be typically 0.7 mm to 1.1 mm and the length of the overlap of the pleats can be selected from a range of about 0.8 mm to 1.3 mm if six pleats are used and about 1.4 mm to 2.5 mm if three pleats are used. For larger balloon diameters, six pleats may be used in order to offset excessive torsional forces and durability of the expandable structure during operational conditions.

Balloon catheter configuration in which the length of the fold overlap is equal to or less than the distance between adjacent struts can also be used to optimize drug scraping. For example, the ratio between fold overlap and the distance between adjacent struts can be 1:1.5, 1:1, or 1:0.

For example, balloon with diameters of 6 mm and 6 pleats, the ratio between fold overlap and the distance between adjacent struts can be 1:0.7.

Referring now to the drawings, FIGS. 1A-3E illustrate embodiments of the present balloon catheter which is referred to herein as device 10.

Device 10 includes a catheter shaft 12 attached to an inflatable balloon 14. Catheter shaft 12 can be up to 150 mm in length and 0.5 mm to 1.5 mm in external diameter. Catheter shaft 12 can include a lengthwise guidewire lumen for accommodating a guidewire 16 and a conduit for inflation of balloon 14. Balloon 14 can be fabricated from non-compliant, semi-compliant or compliant materials such as polyethylene, Nylon, Pebax or polyurethane at various lengths and final (inflated) diameters depending on the intended use. Examples of device 10 can include a balloon having a length between 10 mm to 40 mm for coronary applications and 20 mm to 300 mm for peripheral applications and an inflated diameter between 1.5 mm to 10 mm.

Balloon 14 can be bonded thermally or glued using an adhesive to over the catheter shaft and attached to the inflation conduit running the length of catheter shaft 12.

Device 10 further includes an expandable structure 18 that is constructed from a plurality of radially expandable rings 20 (e.g., up to 16) and a plurality of axial struts 22 (e.g., 4 or more). Expandable structure 18 can include any number of rings 20 and struts 22 depending on balloon 14 length and diameter.

The number of axial struts 22 may increase as the diameter of the balloon 14 increases. For example the balloon 14 shown in FIGS. 1A-1D may be 3 mm in diameter and 20 mm in length. The expandable structure 18 may include ten expandable rings and four axial struts. The number of axial struts may be four for balloons with diameter of 2 mm to 4 mm and six for balloons with diameter of 4.5 mm to 6 mm. The number of expandable rings 20 is proportional to the balloon length. As the balloon lengthens, the number of expandable rings 20 increases. For example a balloon with 3 mm in diameter and 40 mm in length may include twenty expandable rings. The number of expandable rings 20 is also proportional to the balloon diameter, but this time the number of expandable rings 20 is smaller when the diameter is higher. For example a balloon 4 mm in diameter and 20 mm in length can be covered by an expandable structure having 8 expandable rings, and a balloon 4 mm in diameter and 40 mm in length can be covered by an expandable structure having 16 expandable rings.

Expandable structure 18 can be manufactured using techniques known in the art such as laser cutting of a Nitinol tube and electropolishing to produce smooth surfaces and edges radiuses.

As is shown in FIG. 1A, rings 20 can include undulations (e.g., S-shaped regions) for enabling rings 20 to radially expand. Similarly, struts 22 can also include such undulating regions for enabling the struts to lengthen during balloon inflation. In both the rings and struts, such undulating regions determine the extent of radial expansion and lengthening so as to accommodate for balloon inflation and constrain the balloon.

Rings 20 and struts 22 define openings 24 (one opening framed for emphasis in FIG. 1D) in expandable structure 18 through which balloon regions 26 protrude following inflation. FIGS. 1B-D illustrate various stages of inflation and show linearization of rings 20 and struts 22 as well as formation of protruding balloon regions 26 (pillows, best seen in FIG. 1D).

As is mentioned hereinabove, the distance (D, FIG. 1D) between adjacent struts 22 of an expanded expandable structure 18 is selected in order to maximize drug scraping. Such a distance can be greater than or equal to about 0.4 mm and/or less than or equal to about 1.1 mm, such as about 0.4, 0.5, 0.6, 0.7, 0.8, 0.9, 1.0 or 1.1 mm.

Device 10 further includes a coating 30 that can incorporate a composition such as an antiproliferative drug. Coating 30 can cover the balloon surface or the balloon surface and the struts and rings.

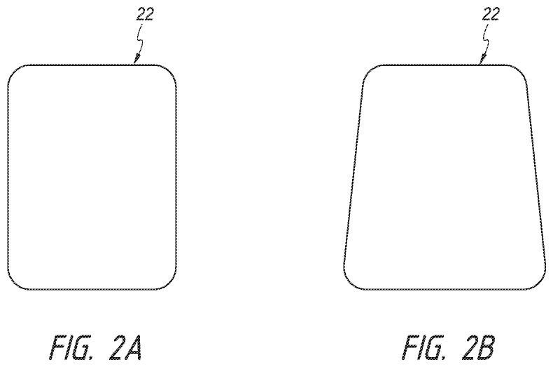

As is shown in FIGS. 2A-2B, struts 22 are fabricated with a unique profile (cross section) in order to enhance scraping of the balloon coating without damaging (tearing) the balloon wall. Such a profile is preferably multi-sided, such as 4-sided (e.g., rectangular, square, trapezoid etc.). FIG. 2A illustrates a rectangular profile while FIG. 2B illustrates a trapezoid profile (with the base positioned to contact the balloon surface).

Such a profile is preferably 4 sided (e.g., square, rectangular, trapezoid) with round edges having a radius of curvature of at least about 0.01 mm and/or less than or equal to about 0.05 mm, such as about 0.01, 0.02, 0.03, 0.04 or 0.05 mm.

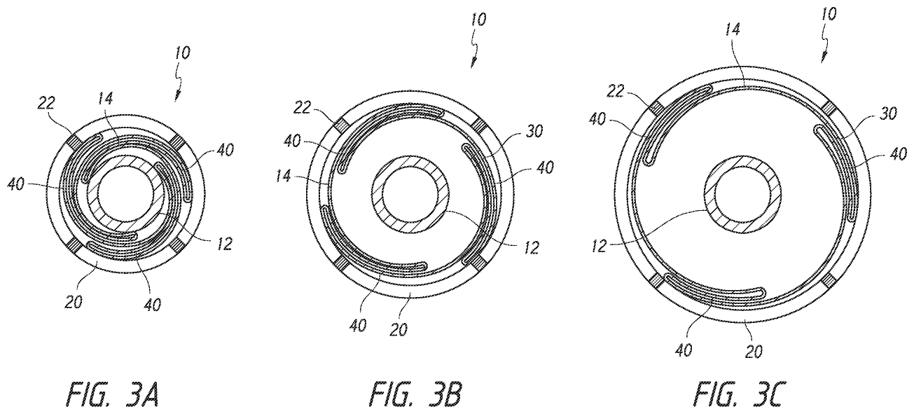

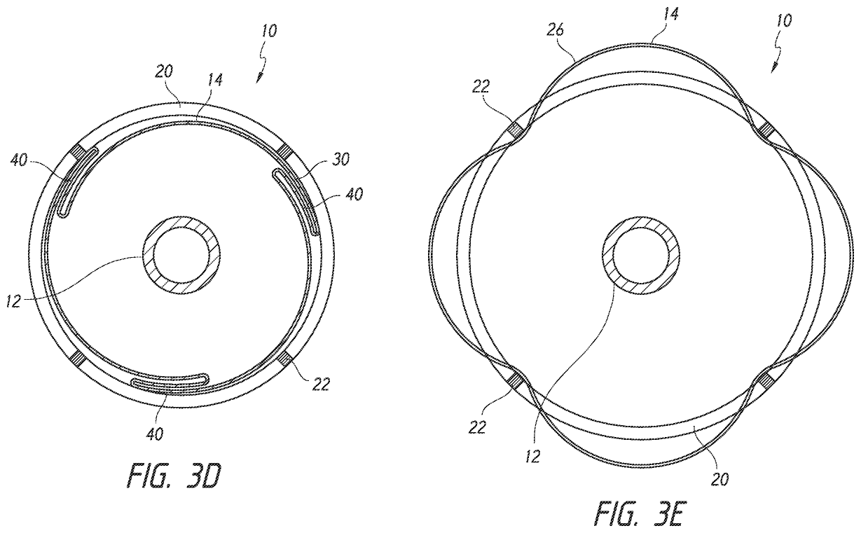

FIGS. 3A-3E illustrate unfolding of balloon 14 during inflation that results in scraping of coating 30 from balloon surface 26.

When packed for delivery, balloon 14 is configured with pleated folds 40 (three shown) that overlap the balloon surface (folded against balloon surface) beneath the expandable structure 18 (see FIG. 3A). As balloon 14 inflates, pleated folds 40 unfold and rotate and thus move against struts 22. Such movement scrapes coating 30 off balloon surface 26 thereby releasing the composition at the site of treatment. In the case of angioplasty, release of the active pharmaceutical ingredient(s) (e.g., Paclitaxel, Sirolimus) and delivery thereof to the arterial wall can reduce or prevent restenosis following angioplasty. In order to maximize scraping, balloon 14 is folded with a low number of pleats (e.g., three pleats). As the number of pleats decreases, the length of the fold increases.

When the balloon is folded with a low number of pleats each pleat is relatively long and therefore when these longer pleats expand and unfold they have a longer tangential travel against the struts.

FIGS. 4A-5B illustrates the relationship between the distance between struts 22 and the overlap length of the pleats 40.

FIG. 4A illustrates a cross section of a device 10 having a diameter of 3.0 mm and folded with six pleats 40, the overlap of each fold is about 0.5 mm.

FIG. 4B illustrate a cross section of a device 10 having a diameter of 3.0 mm and folded with three pleats 40, the overlap of each fold is about 1.0 mm.

FIGS. 4C and 4D illustrate the device 10 of FIGS. 4A and 4B (respectively) and show that the distance between struts 22 is about 0.75 mm. The number of pleats 40 has minor effect on the outer diameter of the folded balloon and therefor the distance between struts 22 is the same for both three and six pleats. As a result, the ratio between folds overlap to the distance between struts in this example is 1:0.75 for the three pleat balloon and 0.5:0.75 for the six pleats balloon.

FIGS. 5A and 5B illustrate a cross section of a device 10 having a diameter of 6.0 mm and folded to form six pleats. These figures show that the folds overlap is about 1.3 mm and the distance between struts is about 0.9 mm. As a result the ratio between folds overlap to the distance between struts is this example is 1.3:0.90, which is equal to 1:0.70.

As is mentioned hereinabove, device 10 of the present invention can be used to deliver a composition to any biological vessel. When utilized in an angioplasty procedure, device 10 is used as follows.

Device 10 is delivered via an access port in the artery, typically a femoral or radial artery, over a pre-positioned guide wire and guided to a coronary or peripheral lesion site.

During the delivery stage the drug coating over the balloon surface is protected from drug loss to blood contact by the expandable structure.

The balloon is then inflated at the lesion site to expand the lesion and deliver the drug to the site. During balloon expansion the balloon pleats unfold underneath the expandable structure, scraping/wiping the drug coating from the balloon surface and allowing it to be pressed into the blood vessel wall. The balloon is held inflated for sufficient time (seconds to minutes) to facilitate drug delivery to the lesion and arterial wall.

The balloon is then deflated and removed and the expandable structure is compressed against the balloon folds to protect the balloon from any residual drug loss during removal.

### Methods of Coating

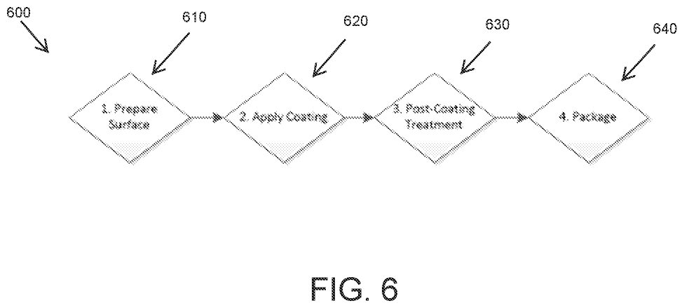

Processes for coating a distal portion of a balloon catheter to yield a drug-coated balloon are disclosed. The coating may include one or more active pharmaceutical ingredients (APIs) and one or more excipients, dissolved in one or more solvents, for example any of the combinations described herein. The solvents typically used are volatile, to reduce or minimize drying time. The processes described herein provide a uniform and repeatable coating over a surface that is non-cylindrical and/or is comprised of more than one material and may be applied to any of the balloon catheters described herein. The coating process 600 may include one or more of the phases shown in FIG. 6.

The processes may be performed on a distal portion of a balloon catheter. In some processes, the distal portion of the balloon catheter may include an inflatable member (e.g., balloon) and at least one additional material or secondary structure that forms a non-cylindrical surface and/or a surface having more than one material. The secondary structure may be another structure that is placed, mounted, or bonded to the balloon. For example, the secondary structure may be a patterned structure resembling a stent with circumferential and/or longitudinal members, such as any of the constraining or expandable structures 18 described above. The secondary structure may include a filament, chord, wire, braided, or coiled structure.

The balloon may include thermoplastic polymers or PET, polyester, Pebax, polyurethane, and/or silicone. The balloon and the secondary structure can include different materials or the same material(s). In some embodiments, the additional material or secondary structure may include a metallic material such as stainless steel, cobalt-chromium, titanium, and/or nitinol. In other embodiments, the additional material or secondary structure may include a polymeric material.

The coating process 600 may include one or more of the below described phases. Any one or combination of these phases may make take place prior to or following the application of the secondary structure or additional material to the balloon.

The coating process 600 may include a surface preparation phase 610. Preparing the surface to be coated can include one or more surface treatments, allowing for an improved interface to form between the coating and balloon catheter surface in subsequent steps. Treatment may include cleaning the surface, removing a layer of material to expose fresh surface beneath, and/or modifying the existing layer.

Immersion or application of a solvent to the surface of the balloon allows for dissolution or rinsing of materials on the surface. For example, solid state (dry) cleaning is a nonabrasive, residue-free method of ensuring the surface of the balloon is accessible, without the presence of organic or hydrocarbon residues. In one example, carbon dioxide may be sprayed or otherwise applied to a surface the balloon. The carbon dioxide changes the surface energy and wetting properties of the balloon surface allowing the coating composition to better adhere to the balloon surface.

Plasma treatment may be used alone or in combination with other surface treatments to expose a fresh surface or activate the surface prior to coating. Activation allows for increasing the surface energy of the surface. On nylon, this results in a super-hydrophilic state, allowing a solution (e.g. the API-containing formulation) to wet the surface of the distal portion of the balloon catheter during the coating process. This results in minimization of any meniscus formation at the interface of 2 or more materials and allows for a more distributed film over the surfaces. The plasma treatment can be done using inert gas, low pressure plasma. Alternatively, the plasma treatment can be performed using atmospheric plasma systems.

The coating process 600 may include a coating application phase 620. The coating application phase 620 may include the application of one or more layers, with at least one of the layers applied with the goal of having a defined amount of the API distributed uniformly and repeatedly over the working length of the distal portion of the balloon catheter. This can be performed using a variety of methods include dispensing an aliquot over the surface, dip coating, or spray coating. For example, the coating may be applied using a single-pass aliquot method, in which the distal portion moves past the dispensing source once while the distal portion is rotating. The linear motion (speed), linear length (distance), dispensing volume, dispensing speed are controlled in this process. A lumen is placed at or near the distal portion to dispense a pre-formulated coating solution and the coating application process is initiated.

In some methods, the balloon may be only partially inflated prior to coating. For example, the balloon may be pressurized within a sheath prior to coating to prevent the balloon from fully opening. The partial inflation may increase a diameter of the balloon compared to an uninflated balloon by at least about 10% and/or less than or equal to about 50%, for example between 10% and 20% or between 40% and 50%. To inflate the balloon, the balloon may be inflated to a pressure between 10 psi and 35 psi, for example between 10 psi to 20 psi or between 25 psi to 35 psi or up to 15 psi, depending on the size. Thereafter, the pressure may be reduced to less than or equal to about 5 psi or less than or equal to about 2 psi. A stopcock on the catheter may be closed to maintain the pressure within the balloon.

In other methods, the balloon may be fully inflated to expose an entire outer surface of the balloon to the coating. When the expandable structure 18 is mounted over the balloon, isolated balloon regions may protrude from openings in the expandable structure to be coated. The balloon may be inflated to a pressure between 10 psi and 50 psi, for example between 10 psi to 20 psi or between 25 psi to 35 psi, depending on the size. When fully inflated, a diameter of the balloon may increase compared to an uninflated balloon by at least about 300% and 400%, for example between 300% and 325%, between 325% and 350%, between 350% and 375%, or between 375% and 400%. A stopcock on the catheter may be closed to maintain the pressure within the balloon.

The coating process 600 may include a post-coating treatment phase 630. Post-processing of the coating is a secondary process that is performed on the coated balloon to establish the final configuration. It is an optional process secondary to the coating, by which the coated surface is modified to homogenize or transform the surface impacting performance. The post-coating treatment phase 630 may include a treatment using a solvent based system used to convert the API into a homogeneous solid state form (e.g. ensure all material on the surface is in a specific state or polymorph). Alternatively, it could be used to remove a soluble secondary component to increase the surface area of a primary molecule to ensure it is amenable to physical transfer to the surface of the artery. Post-processing can be done by immersion in a solvent or deposition of a solvent to transform the API or expose the API. Alternatively, this can be done by developing within a solvent vapor chamber. In some methods, the post-coating treatment phase 630 could include water dipping to remove the excipient. This may also hydrate the API to obtain the desired polymorphic structure.

The coating process 600 may include a packaging phase 640. Packaging is a process by which the device is put into its final configuration prior to its final processing and transport to the location of use. Prior to packaging, the balloon may be deflated. When deflated, the balloon may form folds beneath the secondary structure. The configuration of the folds may resemble any of the fold patterns described herein.

In some embodiments, a balloon catheter may be assembled having a distal portion with a folded balloon and an expandable structure fixedly mounted over the folded balloon. The folded balloon may include a polymeric material such as nylon. The expandable structure may include a different material, for example a metallic material such as nitinol.

The distal portion of the balloon catheter may be coated using one or more of the following steps:

In the surface preparation phase 610, the surface of the balloon may be prepared with solid-state carbon dioxide. A CO₂ composite spray generator may be used. The CO₂ output pressure may be set to at least about 1250 psi and/or less than or equal to about 1350 psi. The propellant pressure may be set to at least about 50 psi. The balloon catheter may be placed within a block mount with the distal portion exposed. The nozzle may be positioned at a distance within about one inch and at an angle of about 45 degrees relative to the balloon catheter. The distal portion of the balloon catheter may be exposed to a steady stream of CO₂ from the nozzle, after inflation to a pressure between 2 psi and 50 psi, allowing the balloon to be partially or fully inflated. For a partially inflated balloon, the pressure may be less than or equal to about 10 psi, for example, less than or equal to about 5 psi or less than or equal to about 2 psi. For a fully inflated balloon, the pressure may be at least about 10 psi and/or less than or equal to about 50 psi, for example between about 10 psi and about 35 psi.

The process may include activating the surface by exposure to low-pressure, inert plasma with the balloon in the inflated state. For example, an Argon plasma may be applied at pressures less than 1 kPa or less than 0.1 kPa. Plasma treatment may be performed on the distal portion of the balloon catheter in the inflated state, so that the surface to be coated is exposed. This can be achieved by using a low pressure gas (e.g. air) to inflate the balloon (3-35 psi). In some cases, slightly higher pressure may be required to inflate the balloon initially (10-50 psi), followed by reduction to the appropriate range. Plasma may be applied for at least 1 min or at least 5 min.

In the coating application phase 620, the process may include depositing liquid formulation of the coating on the surface of the distal portion using an aliquot method. In this case, the distal portion may be rotated at a constant speed (20 rpm) and also linearly advanced relative to the dispensing nozzle (2-10 mm/sec) using a single pass. The liquid formulation may be dispensed at a constant rate, for example at a rate between about 50 uL/min and about 1000 uL/min.

In the post-processing phase 630 of the coating, the applied coating may be immersed for 75 min in a quiescent, aqueous solution at 35 C to ensure the API is in the correct polymorphic form. This results in removal of 25%-75% of a relatively hydrophilic excipient, with less than 5% loss of the hydrophobic active pharmaceutical ingredient.

After the post-processing phase 630, the balloon may be deflated. The balloon may re-fold into a folded configuration beneath the expandable structure based on the interaction between the expandable structure and the balloon during deflation.

### Terminology

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±10%). For example, “about 0.04 mm” includes “0.04 mm.”

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of manufacturing a drug-coated balloon catheter, the method comprising:
mounting an expandable structure over a balloon of the balloon catheter;
surface treating the balloon;
inflating the balloon with the expandable structure mounted over the balloon;
coating the inflated balloon with a composition comprising an active pharmaceutical ingredient and an excipient; and
deflating the balloon to form a plurality of pleated folds beneath the expandable structure.

2. The method of claim 1, wherein mounting the expandable structure over the balloon occurs prior to surface treating the balloon.

3. The method of claim 1, wherein surface treating the balloon comprises spraying carbon dioxide on a surface of the balloon.

4. The method of claim 3, further comprising plasma treating the surface of the balloon.

5. The method of claim 4, wherein applying carbon dioxide to a surface of the balloon occurs prior to plasma treating the surface of the balloon.

6. The method of claim 1, wherein inflating the balloon comprises fully inflating the balloon.

7. The method of claim 1, wherein inflating the balloon comprises inflating the balloon until isolated balloon regions protrude through openings in the expandable structure.

8. The method of claim 7, wherein coating the inflated balloon comprises coating the isolated balloon regions.

9. The method of claim 1, further comprising coating the expandable structure.

10. A balloon catheter comprising:
a balloon coated with a composition; and
an expandable structure mounted over the balloon, the expandable structure comprising a plurality of axial struts crossing a plurality of radially-expandable rings to form a plurality of openings,
the balloon catheter configured to transition between a collapsed configuration and an expanded configuration,
wherein, in the collapsed configuration, the balloon comprises a plurality of pleated folds beneath the expandable structure,
wherein, in the expanded configuration, isolated balloon regions protrude through the openings in the expandable structure, and
wherein the expandable structure is configured to scrape the composition from the balloon as the balloon transitions from the collapsed configuration to the expanded configuration.

11. The balloon catheter of claim 10, wherein a length of overlap of each of the plurality of pleated folds is less than a distance between adjacent axial struts of the plurality of struts.

12. The balloon catheter of claim 11, wherein the length of overlap of each of the plurality of pleated folds is 50% to 80%, inclusive, of the distance between the adjacent axial struts of the plurality of struts.

13. The balloon catheter of claim 10, wherein each of the plurality of axial struts has a cross-section with rounded corners.

14. The balloon catheter of claim 13, wherein a radius of curvature of the rounded corners is selected from a range between 0.1 mm to 0.5 mm, inclusive.

15. The balloon catheter of claim 10, wherein each of the plurality of axial struts has a four sided cross-section.

16. The balloon catheter of claim 10, wherein the composition includes an anti-proliferative drug.

17. The balloon catheter of claim 10, wherein, in the collapsed configuration, the balloon includes between two to six pleated folds, inclusive.

18. The balloon catheter of claim 10, wherein a distance between adjacent axial struts of the expandable structure is selected from a range of 0.4 mm to 1.1 mm, inclusive, when the balloon catheter is in the collapsed configuration.

19. The balloon catheter of claim 10, wherein each axial strut has a width selected from a range of 70 to 90 microns, inclusive, and a height selected from a range of 80 to 120 microns, inclusive.

20. A balloon catheter comprising:
an expandable structure mounted over a balloon,
said expandable structure comprising a plurality of axial struts crossing a plurality of radially-expandable rings for constraining said balloon such that isolated balloon regions protrude through openings in said expandable structure when said balloon is inflated,
wherein each of said axial struts has a four-sided cross section and rounded corners.

21.-37. (canceled)
